(19) United States
(12) Patent Application Publication
(10) Pub. No.: US 2024/0296539 A1
(43) Pub. Date: Sep. 5, 2024

(54) VISION SYSTEM

(71) Applicant: Randox Laboratories Ltd., Crumlin, Northern Ireland Antrim (GB)

(72) Inventors: Deepesh UPADHYAY, Northern Ireland Antrim (GB); Peter FITZGERALD, Northern Ireland Antrim (GB); Ivan MCCONNELL, Northern Ireland Antrim (GB)

(73) Assignee: Randox Laboratories Ltd., Crumlin, Northern Ireland Antrim (GB)

(21) Appl. No.: 18/572,687

(22) PCT Filed: Jul. 1, 2022
(86) PCT No.: PCT/GB2022/051708

§ 371 Date: Dec. 20, 2023

(30) Foreign Application Priority Data
Jul. 2, 2021 (GB) 2109635.9

(51) Int. Cl. G06T 7/00; H04N 23/56; H04N 23/695

(52) U.S. Cl. CPC G06T 7/0004; H04N 23/56; H04N 23/695; G06T 2207/30072

(57) ABSTRACT

There is provided a vision system for assessing defects on biochips and a corresponding method. The vision system comprises an imaging region in which a biochip sheet including at least one biochip is locatable in use: an imager arranged in use to image at least a portion of the imaging region, wherein, when the biochip sheet is located in the imaging region. the portion includes at least a face of the biochip sheet: and an illumination source arranged in use to direct illumination on to the face the biochip sheet. thereby illuminating defects on the at least one biochip when a mask material layer is present on the at least one biochip and when a mask material is absent and allowing the defects to be included in the imaged at least a portion of the imaging region.

### FIELD OF THE INVENTION

The present invention relates to vision systems that are typically used for conducting quality control on biochips fabricated on a production line and methods for conducting the quality control.

### BACKGROUND

Use of fabricated arrays of molecules in the detection and characterisation of analytes is well known. For example, fabricated arrays of polynucleotides are used widely in DNA sequencing procedures and in hybridisation studies for the detection of genetic variations in a patient. Immunoassays are also well known for detecting analytes, such as specific proteins or other binding agents, through their properties as antigens or antibodies.

Micro-array substrates typically include a supporting material comprising a plurality of discrete reaction zones located in spatially distinct areas on the substrate. The provision of multiple reaction zones allows simultaneous testing of multiple analytes or biomarkers in a sample. Micro-arrays are important laboratory tools, not only allowing a more comprehensive analysis of a patient's condition but also saving the time and cost associated with other laboratory tests. Typically, micro-arrays are manufactured by depositing or “spotting” molecules or molecular fragments, also referred to as reagents, on to the substrate to form an array of reaction zones. In order to obtain an acceptable quality of data, the spotting should be uniform so that the spots are of the same size and shape.

In order for accuracy and precision of data obtained from currently available micro-arrays to be improved to allow the micro-arrays to be more effective in both research and clinical settings, a micro-array with a coated substrate as disclosed in EP 3377900 A1 was developed. This provides a coating on the substrate in the areas not occupied by the discrete reaction zones. The coating is typically inert and is a dark colour, which provides a high contrast with the un-coated discrete reaction zones, since the substrate is typically a light colour.

The micro-arrays are able to be fabricated as biochips in a sheet of biochips. The biochips in each sheet undergo a spotting process to deposit the relevant materials on to the discrete reaction zones. The spotting process can provide imperfect results however. As such, to ensure the spotting on the biochips of each biochip sheet is of sufficiently high quality, a quality control procedure is implemented.

Each biochip sheet typically has a ten by ten grid of biochips on it. Known systems, such as the one shown in FIG. 1, allow quality control to be conducted by imaging the biochips on the sheet. This is achieved by backlighting the biochip sheet to allow light to pass through the biochip sheets into a wide angle lens connected to a CCD (charge-coupled device). This allows the whole width of a biochip sheet to be imaged in a single image, and means that, by moving the biochip sheet, images of all the biochips on the sheet can be obtained by capturing only two images.

While this process allows highly effective quality control of biochips that do not have coatings, this process is less effective for providing quality control of biochips with coatings, for example screen-printed coatings. This is because the passage of light through the biochips is blocked by the coating in all parts of each biochip other than the discrete reaction zones.

Artefacts, such as spots, can be deposited outside of the discrete reaction zones, and other artefacts, such as scratches, can also be present outside of the discrete reaction zones. As such, an inability to image artefacts outside of the discrete reactions zones significantly lowers the effectiveness of the quality control on coated biochips.

This may be resolvable by using an imager capable of capturing higher quality images, such as an imager with a higher dynamic range and/or greater sensitivity.

However, this increases the complexity and therefore the expense of the camera, which is already a high cost component in any quality control vision system. This is therefore undesirable.

There is therefore a need to improve the effectiveness of quality control able to be carried out on coated biochips, while maintaining suitable effectiveness of quality control on non-coated biochips and without requiring a higher quality imager to be used.

### SUMMARY OF INVENTION

According to a first aspect, there is provided a vision system for (i.e. suitable for) assessing defects on biochips (such as biochips fabricated on a production line), the vision system comprising: an imaging region in which a biochip sheet including at least one biochip is (removably) locatable in use; an imager arranged in use to image at least a portion of the imaging region, wherein, when a biochip sheet is located in the imaging region, the portion includes at least a face of said biochip sheet; and an illumination source arranged in use to direct illumination on to the face of said biochip sheet, thereby illuminating defects on the at least one biochip when a mask material layer is present on the at least one biochip and when a mask material is absent and allowing the defects to be included in the imaged at least a portion of the imaging region.

We have found that by directing the illumination on to the same face as is being imaged allows artefacts on each imaged biochip that are outside of the discrete reaction zones to be identifiable in the image that is producible using the imager. This is due to the light recorded at the imager to form an image being reflected off the biochip face, instead of passing through the biochip as occurs in known systems, thereby allowing the whole face of a biochip to be imaged. Accordingly, this allows artefacts to be identified on coated and non-coated biochips. Additionally, no enhanced imager is required in order to achieve this.

By the term “imager”, we intend to mean an apparatus that includes camera, such as a CCD or CMOS camera. The camera may also have a lens attached thereto in order to allow imaging to take place by focusing light incident on one end of the lens on to a sensor chip of the camera. The camera may be operable locally, such as from a user interface on the camera itself, but is typically operated remotely, such as from a computer program or software operated from somewhere outside of the camera.

The imager is intended to image at least a portion of the imaging region by receiving illumination from the illumination source that has been reflected from the biochip sheet present in the at least a portion of the imaging region.

We intend the face of the biochip sheet to include a face of each biochip of the biochip sheet that is to be imaged. This thereby allows a face of one, a plurality or all the biochips of the biochip sheet to be imaged by the imager.

The imager may be arranged in use to image the portion by imaging individual sections of the portion sequentially such that when the biochip sheet includes a plurality of biochips, the imager images faces of a subset of the plurality of biochips when imaging each individual section of the portion. This allows a lower quality imager to be used while retaining the same quality and/or spatial resolution in an output. This is because the field of view of the imager is reduced from a field of view that encompasses the whole biochip sheet, thereby enhancing the spatial resolution since less area is imaged.

In order to achieve sections of the biochip sheet being imaged, or for other reasons, the biochip sheet may be moveable automatically or manually to re-position the biochip sheet. Typically however, the imager is moveable in use. This allows camera/imager settings, orientation and field of view to be fixed while still allowing movement between sections. Additionally, differences in distortion effects that would be caused by shifting the orientation of the imager relative to the biochip are minimised. Further, this means the biochip sheet is able to be kept stationary while being imaged. This reduces the likelihood of contamination, damage or incorrect positioning of the biochip sheet between imaging occasions.

Movement of the imager may be manual or automatic through any suitable mechanism. This may include a robotic arm or other single or multi-dimensional mechanisms. The imager may be connected to a moveable stage. As noted above, this avoids or reduces a need to move the biochip sheet during an imaging run (i.e. during a period during which a single biochip sheet is being imaged). The use of a stage also provides a reference frame for movement of the imager allowing easily repeatable movements and reliable positioning of the imager. This enhances a quality control process as variation between imaging occasions and between imaging runs is reduced.

Typically, the imager is moveable in two dimensions, such as in a plane (generally, i.e. approximately) parallel to the face of the biochip sheet. By allowing the imager to be moveable in two dimensions, the field of view of the imager can be reduced. This reduces the needed image quality and/or chip resolution for recorded images to achieve the same result in terms of spatial resolution of an output. When the imager is connected to a moveable stage, the stage may be a (XY) gantry.

The biochip sheet is locatable in use on a conveyor. This allows the biochip sheet to be moved into position for analysis while providing easy access to a citing location for the biochip sheet when being loaded for assessment.

Movement of the imager may be fully or partially automated. Typically though, the position of the imager is adjustable by the user. This allows a user to set a position of imager relative to biochips to be imaged, providing the ability to adjust or fine tune positioning and make corrections should any unintended movement occur.

The imager may be arranged in use to provide an image to a user, the image may show the content of the field of view of the imager and a reticule in a fixed position relative to the imager thereby allowing the user to determine the position of the imager relative to the content in the field of view of the imager. This allows a user to accurately, reliably and repeatably position the imager relative to the biochip sheet, for example to calibrate the positioning of the imager relative to the biochip sheet.

As an alternative, the position of the imager relative to the content in the field of view of the imager may be automated, such as by the vision system being arranged to align a point in the field of view with a marker (for example a fiducial marker) on a biochip or on the biochip sheet. This would allow automated calibration of the imager position.

When imaging the biochips, the imager may be arranged in use to travel along a movement path, travel along the movement path causing the imager field of view to be moved to each biochip to be imaged. This allows the imager to follow a pre-defined course when imaging biochips. This makes the movement repeatable and reliable. As long as the field of view is able to be moved across the biochip sheet so as to allow the field of view to coincide with each biochip that is to be imaged during travel along the movement path, this also allows the field of view to be kept to a minimum size allowing a higher spatial resolution to be achieved. It may be considered undesirable to reduce the field of view size since this lengthens the movement path needed to move the field of view over each biochip that is to be imaged. However, we have found that the improvement in spatial resolution allows a lower quality imager to be used while still allowing an improved spatial resolution to be achieved. As such, this outweighs the effect of reducing the size of the field of view.

The movement path may be pre-defined or may be programmed by a user. Typically however, the vision system is arranged in use to calculate the movement path based on a start position and an end position and a value indicative of a number of biochips to be imaged based on the biochips being arranged in an array pattern. This allows the path to be accurately determined by the system reducing the likelihood of human error, thereby making path calculation more reliable. The start position and/or end position may be input by a user. The calculation of the movement path may also be based on the number of biochip faces that fit within a field of view of the imager at the distance from the imager at which the biochip faces are located.

By the phrase “a value indicative of a number of biochips to be imaged”, we intend to mean an input from which it is possible to derive the number of biochips to be imaged. For example, the input may be coordinates on a biochip sheet representing a start position and/or end position for the imager, the coordinates corresponding to a specific biochip in the sheet of biochips. As such, the value may be two values, such as two coordinates, one for the start position and one for the end position. This may be provided in addition to (absolute) positions for the start position and end position relative to a zero point for a mechanism arranged in use to move the imager.

The start position, end position and/or value may be providable to the system by automated acquisition, such as by moving the imager over the biochip sheet and conducting analysis to identify these parameters. Alternatively, the start position, end position and value may be pre-programmed into the vision system. Typically however, the start position, end position and value are providable by a user. This gives a user flexibility to determine how many biochips to image.

A number of biochip faces that fit within the field of view may be determined automatically, be pre-programmed or be provided by a user. Typically this is pre-programmed however. By this we intend to mean a determination of how many biochip faces fit within the field of view is carried out. This is intended as corresponding to a determination of the size of the field of view relative to the biochips at the face of the biochip sheet.

The vision system may further comprise an analyser arranged in use to detect artefacts located on an imaged biochip or at least a portion of the biochip sheet based on an image output from the imager. This allows automated quality control of biochips/biochip sheet to be carried out since user input is not required to review images obtained by the imager in order to assess each biochip. The analyser may be a computer programmed to analyse images output by the imager or software able to be operated or run by a computer or processor.

When an analyser is present, the imager may be arranged in use to provide each image to the analyser and/or the analyser may be arranged in use to receive each image from the imager. In other words, the imager may output each image and/or the analyser may receive each image as an input.

The analyser may be arranged in use to detect artefacts by conducting any one of a number of image processing processes, including edge detection. Typically, the analyser is arranged in use to detect artefacts by assessing changes in contrast and/or changes in pixel intensity between adjacent pixels in an image. This provides a simple mechanism for detecting artefacts and therefore minimising processing needs to conduct the detection. The pixel intensity may include (only) grey values.

The analyser may be further arranged in use to apply a region of interest to each imaged biochip or of each at least of portion of the biochip sheet and to detect artefacts (only) within the region of interest. This allows artefact detection in a reduced area further reducing processing power needed to conduct the detection.

Each biochip may include a substrate comprising a mask material layer and a plurality of discrete reaction zones, each discrete reaction zone being an area of the substrate where the mask material is absent, the region of interest applied by the analyser overlapping with at least a part of the mask material layer (and may exclude the discrete reaction zones). In combination with illumination being directed on to a face of the biochip sheet (and thereby on to the faces of biochips of the biochip sheet), this allows scratches and spot detection on coated sections of coated biochips.

The analyser may apply two regions of interest. Each region of interest may be the inverse of other region of interest within an area. One region of interest may include (only) the sections of a biochip between the discrete reaction zones, the other region of interest may include (only) the discrete reaction zones. These regions of interest may be combined and/or there may be one or more further regions of interest thereby allowing the whole biochip face to be included in a single region of interest or for a region of interest to be limited to a smaller portion of the biochip face.

The analyser may be further arranged to output results of the artefact detection for each imaged biochip or each at least a portion of the biochip sheet. This allows a user to take action in response to analysis, such as to remove a biochip from further processing due to poor quality, and/or to adjust a fabrication process in view of the results. The output may be provided on a graphical user interface (GUI) or display at a terminal or on the vision system itself.

An aperture though which the imager is able to obtain images may be located in use within about 40 millimetres (mm) to about 70 mm of the (face of the) biochip sheet when the biochip sheet is located in the imaging region, such as within about 50 mm to about 60 mm of the biochip sheet. This increases the spatial resolution relative to when the aperture is located further from the face of the biochip sheet. The aperture may be a proximal end of a lens to the face of the biochip sheet, an imaging sensor of the imager typically being connected or in optical communication with a distal end of the of the lens to the face of the biochip sheet.

The illumination source may be a light sheet, light panel or light strip, bulb(s) or other source of illuminations. Typically, the illumination source is a ring light located around an aperture (such as the lens aperture referred to in the previous paragraph) though which the imager is able to obtain images. This provides uniform illumination across the field of view of the imager and limits shadows being cast over the field of view.

Based on the relative position of the aperture and the imaging region, the size of the field of view of the imager at the imaging region may be up to five biochips in length and width, such as up to three biochips in length and width. Typically, the field of view of the imager at the imaging region may be up to two biochips in length and up to three biochips in width. The biochips are typically square and are typically up to about 1.0 centimetre (cm) in length and width, such as about 1.0 cm in length and 0.9 cm in width. Each biochip sheet typically has a ten by ten grid of biochips.

The illumination source is typically a light source, which may emit visible light, the visible light emitted may be white light. The illumination may be diffuse, for example due to being passed through and/or reflected off a diffuser or diffused by some other means.

The vision system may further comprise a support arranged in use to support a platen for a biochip sheet, the platen being locatable to position the biochip sheet in the imaging region. Using a platen maintains environmental conditions of biochips on the biochip sheet located on the platen. This conserves at least some of the conditions of the production line allowing quality control to be more representative of the prevailing conditions under which the biochips were fabricated. The platen may be locatable in use on a conveyor arranged in us to move the platen between a loading region and the imaging region.

The support may be further arranged in use to support a rack for biochip sheets, the rack also being locatable to position at least a biochip sheet held at a top of the rack in the imaging region. This provides flexibility to use a rack in the vision system as well as, or instead of, a platen, and also allows imaging of two biochip sheets if a platen and rack are each located on the support with a biochip sheet positioned on each. This also allows a plurality of biochip sheets to be locatable in a rack in the vision system in order to carry out quality control on multiple biochip sheets without removing the biochip sheet holder (i.e. the rack) from the vision system reducing the likelihood of contamination of biochips held in the rack.

The imager may be arranged in use to provide a spatial resolution of between about 10 microns (μm) and about 1 μm, such as between about 5 μm and about 1 μm, such as about 5 μm. Providing a resolution at this levels allows a high level of detail when imaging biochips, which allows scratch detection and spot detection. This enhances the ability identify artefacts present on an imaged biochip.

The spatial resolution the imager mat be arranged in use to provide may be provided by one or more of the relative position of the imager and biochip sheet or imaging region, the imager field of view, and size of a sensor of the imager. Using a combination of these factors to provide the spatial resolution allows a lower cost imager to be used since the spatial resolution does not need to be provided entirely by a lens and sensor arrangement, which are typically the highest cost elements of an imaging system.

According to a second aspect, there is provided a method of imaging biochips suitable for assessing defects on biochips, the method comprising: illuminating, in an imaging region, a biochip sheet including at least one biochip, the illumination being directed on to a face of the biochip sheet; and imaging at least a portion of the imaging region, the portion including at least a part of the face biochip sheet so as to image a face of at least one biochip of the biochip sheet, thereby illuminating defects on the at least one biochip when a mask material layer is present on the at least one biochip and when a mask material is absent and allowing the defects to be included in the imaged at least a portion of the imaging region. The illumination may be provided by an illumination source, such as a ring light.

The biochip sheet may have (only) a single biochip or may have a plurality of biochips. Of the total number of biochips on each biochip sheet, when there are a plurality of biochips, only some (i.e. a subset) of the biochips on each biochip sheet may be imaged as part of the method according to the second aspect. Alternatively, all the biochips on each biochip sheet may be imaged as part of the method according to the second aspect.

The method according to the second aspect may further comprise detecting artefacts located on an imaged biochip by analysing an image of the at least a portion of the imaging region. This may further comprise outputting results of the artefact detection.

Imaging at least a portion of the imaging region may include imaging the face of each biochip to be imaged by moving an imager along a movement path, the movement path being configured to move a field of view of the imager over the face of each biochip to be imaged.

The method according to the second aspect may further comprise calculating the movement path based on a start position, end position and a value indicative of a number of biochips to be analysed based on the biochips to be imaged being arranged in an array pattern.

### BRIEF DESCRIPTION OF FIGURES

An example vision system and example imaging method are described in detail below with reference to the accompanying figures, in which:

FIG. 1 shows a schematic view of a prior art vision system;

FIG. 2A and 2B show images produced using the prior art vision system;

FIG. 3 shows a schematic view of an example vision system;

FIG. 4 shows an example biochip sheet;

FIG. 5A and 5B show example images producible using the example vision system;

FIG. 6A and 6B show example images producible using the example vision system of FIG. 3;

FIG. 7 shows a plot of grey value of a pixel column against pixel position of a portion of the image shown in FIG. 6B;

FIG. 8 shows a sectional view of an example vision system;

FIG. 9 shows an example GUI for an example vision system; and

FIG. 10 shows a flow diagram of an example imaging method.

### DETAILED DESCRIPTION

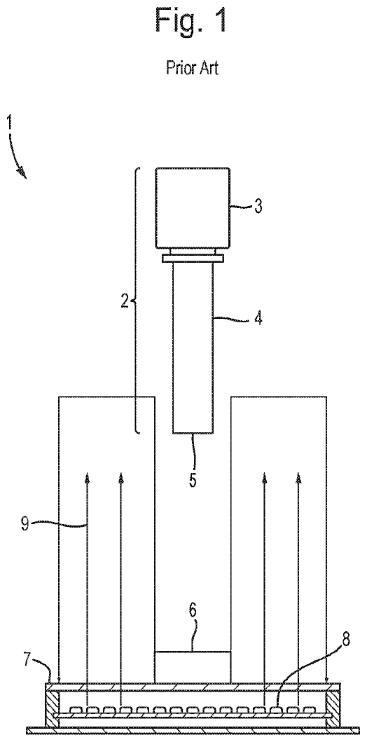

Quality control of biochip spotting has previously been carried out using an arrangement corresponding to the arrangement generally illustrated at 1 in FIG. 1. This includes an imager 2, comprising a camera 3 and lens 4. The lens is connected at one end to the camera and has an aperture 5 at an opposing end.

The lens aperture 5 is orientated towards a biochip sheet comprising biochips 6. The biochips 6 are located on a surface of a linear stage 7 capable of moving the biochips in one dimension.

A light source in the form of LEDs 8 is located underneath the surface of the linear stage 7. As indicated by the arrows 9 in FIG. 1, when in use, the direct light through the surface of the stage on which the biochips 6 are located towards the aperture 5 of the imager 2. Where the biochips are located, the light also passes through the biochips. This illuminates the biochips allowing the imager to obtain images of the biochips and details thereon.

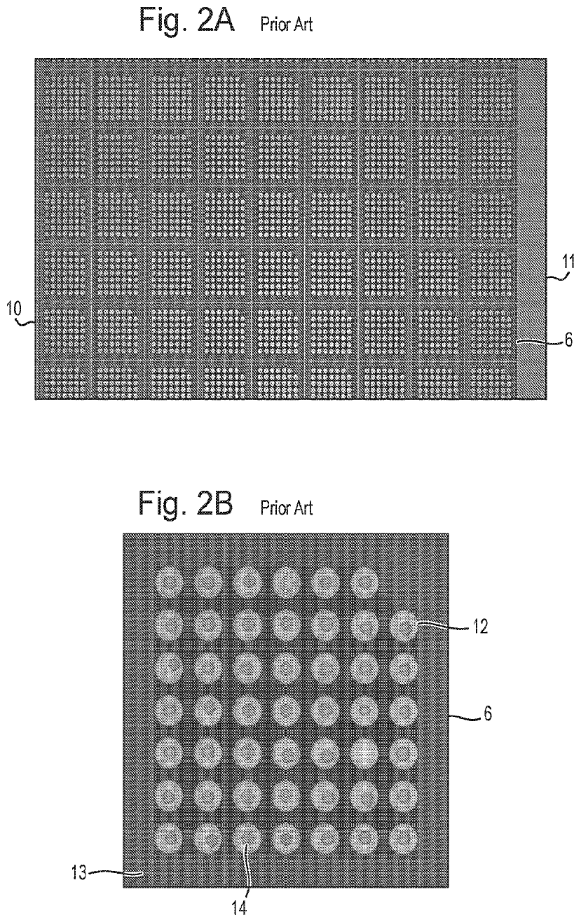

FIG. 2A shows an example field of view 10 of the imager 2 of FIG. 1. This shows a portion of a biochip sheet 11.

The biochip sheet 11 has biochips 6 arranged in a grid with parallel rows and columns of biochips. Typical biochip sheets have a ten by ten grid of biochips. As can be seen from the example in FIG. 2A, the field of view 10 provides visibility of ten columns (the columns being orientated parallel to the length of the page on which the figure is shown), five full rows (the rows being perpendicular to the columns) and two partial rows. This provides a complete visibility of 50 biochips. These are able to be imaged using the imager 2. In order to image the other 50 biochips on the biochip sheet, the sheet is moved on the linear stage 7 shown in FIG. 1 to move the other 50 biochips in the plane of the biochip sheet 11 into the field of view of the imager.

Each biochip 6 shown in FIG. 2A is a coated biochip. FIG. 2B shows a closer view of a single biochip. From this it is easier to see that this example biochip has an array of discrete reaction zones 12. The discrete reaction zones are arranged in rows and columns in the same orientation as the rows and columns of the grid of biochips of the biochip sheet 11.

In the example biochip shown in FIG. 2B, there are seven rows and seven columns of discrete reaction zones with one discrete reaction zone omitted from the grid at one corner. On the biochip shown in FIG. 2B, there is no discrete reaction zone in the top right corner. The rows and columns of discrete reaction zones can also be seen on each biochip of the biochip sheet shown in FIG. 2A.

The example biochips shown in the figures are formed of a ceramic substrate on which there is a mask material layer 13. In this example, the ceramic is predominantly alumina, and is a white ceramic, so is a light colour. The mask material layer is a dark colour. The discrete reaction zones 12 are not covered by the mask material layer, so are exposed ceramic on to which it is intended reagents are placed during production.

In FIG. 2B, spots 14 of reagent in the discrete reactions zones 12 are visible. This is possible due to the light passing though the biochip 6 from the LEDs 8 located underneath the biochip sheet 11 as shown in FIG. 1.

Returning to the camera 3 of the imager 2, in the example shown in FIG. 1, the camera is a CCD camera (such as a FLI Microline ML50100 Monochrome CCD camera). The FLI Microline ML50100 Monochrome CCD camera has a 16-bit analog-to-digital conversion (ADC), back focus of 21.9 mm, temperature range of 45 degrees Celsius (° C.), 11 stop dynamic range, full well capacity of 40.3 ke, 50.1 Megapixels, Peak quantum efficiency (QE) of 61%, a chip with a pixel array of 8,176 by 6,132 pixels, a pixel size of 6 μm and read noise of 12 e. Using this camera, or one with corresponding specifications, when the lens aperture is located about 150 mm to about 200 mm from the upper face of the biochips 6 in the biochip sheet 11 to provide the field of view shown in FIG. 2A, a spatial resolution of about 12 μm is achieved. Additionally, due to the size of the field of view, it requires two images to image all the biochips on a single biochip sheet.

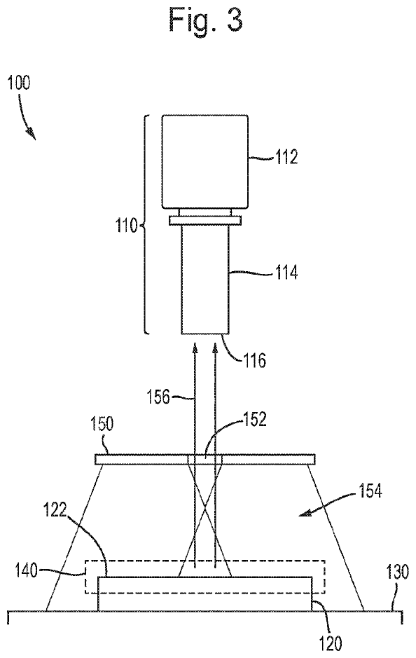

Compared to using the system shown in FIG. 1, we have found that the spatial resolution can be improved, and enhanced quality control of biochips can be achieved while using a low cost imager. This is achieved by using a system such as the vision system generally illustrated at 100 in FIG. 3.

The example vision system 100 shown in FIG. 3 has an imager 110. The imager includes a camera 112 and lens 114. The lens is connected at one end to the camera and has a lens aperture 116 into which light is able to pass at an opposing end.

The lens aperture 116 is orientated to align with the normal to the plane in which biochips 120 are placed that the imager is intended to image, and therefore to be orientated normal to a face of each biochip to be imaged. As such, the lens aperture is above the biochips.

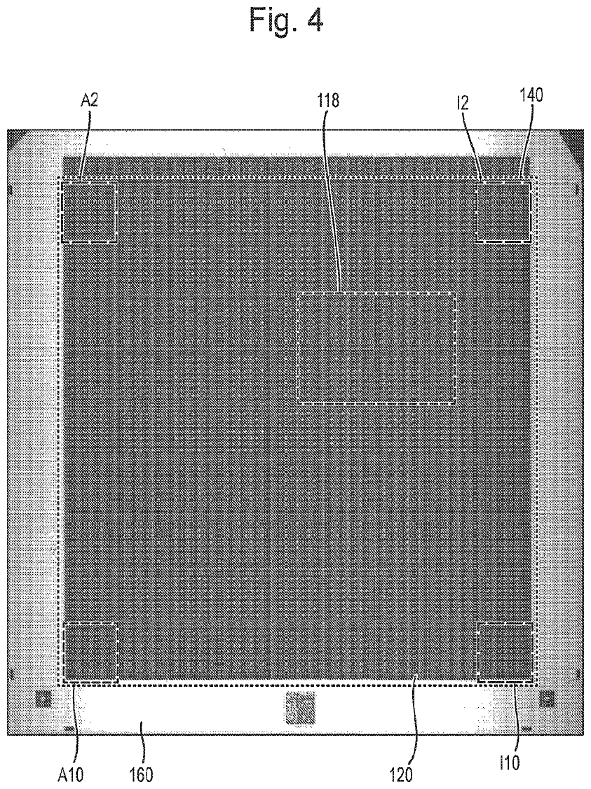

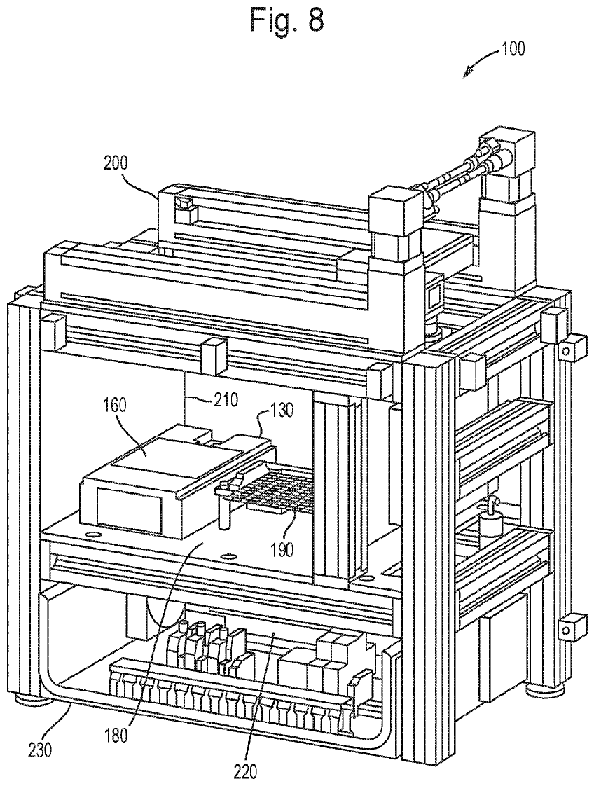

Biochips 120 that are to be imaged form part of a biochip sheet 160 (an example of which is shown in FIG. 4). In some examples, the biochip sheet, and therefore the biochips, is placed on a platen 130. As explained in more detail below (in relation to FIG. 8), one or more biochip sheets are additionally or alternatively able to be mounted in a rack in some examples.

The lens 114 of the imager 110 allows imaging to occur by bringing objects at a certain distance into focus (as is the typical function of a lens). An adequate degree of focus is maintained over a distance range typically referred to as the depth of field. The focus position and depth of field provides an imaging zone 140 within which the imager is able to capture an “in focus” image with the camera 112. When a biochip sheet 160 with biochips 120 mounted on a platen 130 is appropriately located in the vision system 100, this allows a face of the biochip sheet, and therefore a face 122 of each biochip, to be positioned in the imaging zone. In some examples, the platen is loaded on to a conveyor to allow the platen to be moved between a loading location and the imaging zone in a consistent and reliable manner.

In various examples, the conveyor is a conveyor that links a spotting system, such as one that prepares the biochip sheets by spotting reagent(s) on the one or more biochips, and the vision system as well as any further processing systems. This provides transport for a biochip sheet holder, such as a platen. Transporting the biochip sheet holder in this manner allows the conveyor to pass a biochip sheet to the vision system for processing as described herein, and then to transport the sheet away from the vision system.

On being transported away from the vision system, several options are possible. For example, the biochip sheet can be transported to an incubator or moved on for other processing, use or packing by an unloading robot. When a conveyor is used, at least the vision system is typically provided with an enclosure designed to avoid influence from light external to the enclosure, such as by minimising light ingress to the enclosure with features like light-tight seals or other light reduction means.

In order for the imager 110 to be able to capture an image, light from the object to be imaged needs to be recorded by a sensor (not shown) of the camera 112. In this example, the illumination source providing the light for the imaging is a ring light 150.

The ring light 150 has a ring aperture 152 (in this example at the centre of the ring). The ring aperture is aligned with (such as being coaxial with) the lens aperture 116 of the imager 110.

In the example shown in FIG. 3, the imager 110 is positioned with the lens aperture 116 about 50 mm to 60 mm from the biochip faces 122. The vision system shown in FIG. 3, also shows the ring light 150 located about half way between the lens aperture 116 of the imager 110 and the imaging zone 140.

In other examples the distance between the lens aperture 116 and the biochip faces 122 may be different. Additionally, or alternatively, the ring light 150 is able to be located adjacent the lens aperture or another part of the imager 112 or between the lens aperture and the imaging zone 140. This is possible as long as light emitted by the ring light is able to pass (once reflected from a surface) through the ring aperture 152 to the lens aperture.

The ring light 150 is able to emit diffuse light in use. In some examples, the light emitted is white light. The ring light is positioned (approximately) parallel to the faces 122 of the biochips 120 on the platen 130 and is orientated to direct light 154 it emits to towards the imaging zone 140. When biochips are located in the path of the light emitted by the ring light, the light reflects off the faces of the biochips. As indicated by arrows 156 in FIG. 3, at least some of this reflected light passes through the ring aperture 152 to the lens aperture 116 of the imager 110. This light is recorded by the camera sensor to allow imaging of biochip faces to occur.

The imager on an example as shown in FIG. 3 is able to use a camera 112 with a Sony IMX183 CMOS sensor (not shown). This sensor has a (virtual) rolling shutter, a maximum image circle of about 1 inch (2.54 cm). The size of the sensor is about 13.1 mm by about 8.8 mm; and the sensor has a pixel resolution of 5,472 by 3,648 pixels, with an overall resolution of 20 Megapixels. The pixel size of the sensor is about 2.4 μm by 2.4 μm. The frame rate achievable is 17 frames per seconds (fps), and it is a colour sensor instead of a monochrome sensor. In other examples other corresponding sensors can be used, including corresponding monochrome sensors.

In some examples, the ring light 150 is a Moritex Corporation CF-FR series ring light. Other examples use other similar ring lights.

Turning to the biochip sheet 160, an example biochip sheet can be seen in FIG. 4. This shows a face of the biochip sheet, specifically corresponding to the upper surface of the biochip sheet. This face includes the face of each biochip 120 of the ten by ten grid of biochips.

Each biochip 120 is about 1.0 cm by 0.9 cm in width and length (width corresponding to the direction from left to right on the page and length corresponding to the direction from top to bottom of the page). In this example, the distance between the lens aperture 116 and the biochip faces 122 provides a field of view (illustrated in FIG. 4 by the dashed box at 118) capable of being imaged by the camera 112 in a single frame that is about the size of three biochips in width and about two biochips in length.

In the example shown in FIG. 4, the imaging zone is illustrated by the dotted box 140 that encompasses all the full size biochips 120 of the biochip sheet 160. In other examples, the imaging zone may be larger (such as to encompass all or part of one or more further biochip sheets) or smaller, the imaging zone defining the area to be imaged by the imager during use of the vision system. How this is achieved is described in more detail below.

To allow easy identification of the individual biochips 120 in a biochip sheet 160, the grid of biochips is allocated coordinates. As such, since each biochip sheet has biochips arranged in the same grid pattern, the same coordinates can be applied to each biochip sheet to be analysed by the vision system 100. In the examples shown herein, the coordinates are the letters “A” to “I” and numbers “2” to “10”. The letters are applied to each column of the grid such that each column is identifiable by a single letter, and the numbers are applied to each row of the grid such that each row is identifiable by a single number. This allows a single letter and number combination to identify a specific biochip in any given biochip sheet.

In the example shown in FIG. 4, the letter “A” is allocated to the left most column of the grid of biochips 120. The letter identifying each column is incremented by one letter per column from left to right. This causes the right most column to be allocated the letter “I”. In this example, the number “2” is allocated on the top most row of the grid of biochips. The number identifying a respective row is incremented by one per row from top to bottom, which results in the bottom most row being allocated the number “10”. This system of coordinates means the biochips encircled by the long dashed boxes in FIG. 4 at each corner of the grid of biochips are biochip A2 in the top left corner, biochip 12 in the top right corner, biochip A10 in the bottom left corner and biochip 110 in the bottom right corner.

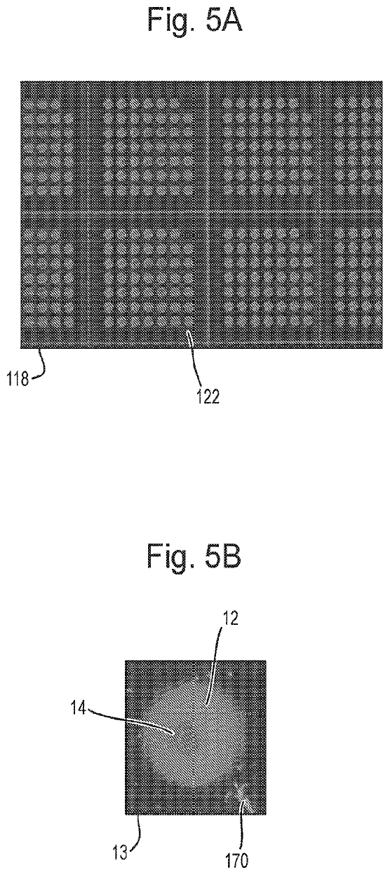

FIG. 5A shows an example image obtainable using the imager 110 when a vision system 100 corresponding to the example vision system shown in FIG. 3 is used to image a biochip sheet 160 corresponding to the example biochip sheet shown in FIG. 4. The image shows the complete field of view 118 of the imager. This includes a plurality of biochip faces 122, specifically this shows all of four biochip faces in a two by two grid and about half of each of four further biochip faces with a half biochip face being shown at the sides of each row.

FIG. 5B shows a zoomed in portion from an image like the one shown in FIG. 5A. This shows the level of detail available in an image obtained with the imager 110 of the vision system according to an aspect disclosed herein.

The image in FIG. 5B shows an area of a biochip face 122 around a discrete reaction zone 12. The discrete reaction zone is shown as a light circle in the middle of the image shown in FIG. 5B. The discrete reaction zone is surrounded by a mask material layer 13.

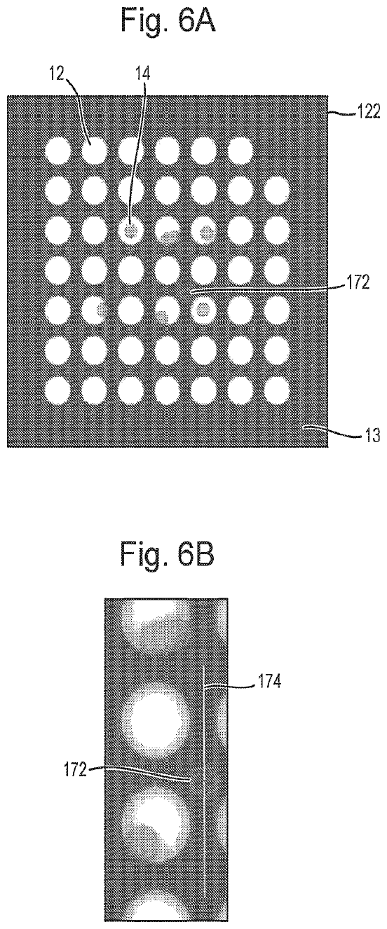

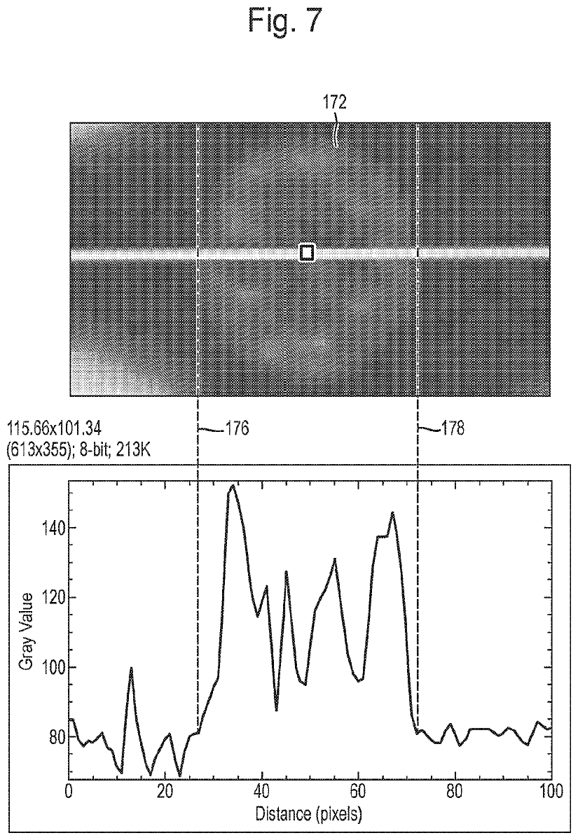

There are two artefacts present in the image shown in FIG. 5B. One artefact is a spot 14 near the centre of the discrete reaction zone 12. The other artefact is a scratch 170 on the mask material layer 13. Each of these artefacts is easily distinguishable and shown clearly in the image. This is because we have found that by using a vision system according to an aspect disclosed herein (such as a vision system corresponding to one of the examples described above in relation to FIG. 3 or later), with the field of view described above and the separate distance between the lens aperture 116 and the biochip faces 122 described above, a spatial resolution of about 5 μm is able to be achieved. This level of spatial resolution allows highly detailed detection of artefacts to be carried out. As shown from FIGS. 6A, 6B and 7, the resolution provides the ability to distinguish between different parts of an individual artefact.

FIG. 6A shows an example biochip face 122 that has been imaged using an imager according to an aspect disclosed herein (such as an arrangement described in relation to FIGS. 3 to 5B). The biochip face has a grid of discrete reaction zones 12 as described above in relation to FIG. 2B and a mask material layer 13 arranged on the non-reaction zone parts of the face. As can be seen in a number of the discrete reaction zones, there are spots 14 of reagent spotted within various discrete reaction zones. Some of these spots are completely within the respective discrete reaction zone and others are partially within the respective discrete reaction zone while also extending partially over the mask material layer. Additionally, in this example, there is one spot 172 located completely outside of a discrete reaction zone due to the spot having been incorrectly located during the process of applying the spot. Each of the spots shown in FIG. 6A is an artefact on the biochip face.

FIG. 6B shows a closer view of the spot 172 located completely outside a discrete reaction zone 12. The spot is formed of circular ring of reagent with an area in the centre of the ring that does not include as much reagent. FIG. 6B also shows an analysis line 174. The pixel value (such as pixel intensity) of the pixels along a portion of this analysis line is shown in the plot of FIG. 7.

The plot in FIG. 7 shows the grey value (marked “Gray Value”) on the y-axis against the distance between two points counted in number of pixels. The plot then shows the grey value for each pixel between those two points along the analysis line 174 shown in FIG. 6B. A portion of that figure is reproduced above the plot to show how the plot and the analysis line align with each other. As can be seen from the reproduced figure, there is a ring-shaped artefact 172. The edges of this ring are marked by dashed lines 176 and 178 in FIG. 7, which extended between the reproduced figure and plot. As can be seen from the plot, the grey value between these dashed lines increases to peaks as the analysis line and the ring intersect and the colour of the pixel at that location is lighter in colour than the mask material 13 colour due to the presence of reagent on the mask material. In various examples, by conducting analysis of changes in grey values and/or contrast artefacts, such as the artefact 172 shown in FIG. 6B and FIG. 7, are able to be identified.

A similar analysis process is able to be applied to identify artefacts wholly contained within a discrete reaction zone 12 and artefacts that are located across the boundary between a discrete reaction zone and the mask material layer. This allows an assessment of the position of reagent spots relative to their intended location to be carried out as well as allowing artefacts to be identified.

In order to obtain images for analysis to detect artefacts, due to the size of the field of view 118, the imager 112 needs to be able to move across each biochip sheet 160 to allow all the biochips 120 on each biochip sheet to be imaged. This is achieved by the biochip sheet 160 being placed within a vision system 100 as shown in FIG. 8. To do this, the biochip sheet, which is typically mounted on a platen 130 during fabrication, is placed on a support 180. It is also possible for a biochip sheet to be held in a rack 190, which can also be placed on the support.

In the example vision system 100 shown in FIG. 8, it is possible to image a single biochip sheet 160, or two biochip sheets. When a single biochip sheet is to be imaged, this may be located on a platen 130 or a rack 190. The platen and rack are each removable from the vision system and can be replaced with another platen and/or rack. When two biochip sheets are to be imaged, these are located adjacent each other on a platen and rack, two platens or two racks. The racks are each able to hold a stack of up to 25 biochip sheets. Since only the top biochip sheet can be imaged, in order to image more than one biochip sheet loaded in the rack, the biochip sheets have to be removed from the rack and re-ordered.

In the example shown in FIG. 8, the support 180 is a shelf. This is located underneath an XY gantry 200, to which the imager 110 and ring light 150 (neither of which is shown in FIG. 8) is attached. This allows the imager and ring light to be moved across each biochip sheet to make imaging possible.

In order to keep noise in the images to a minimum the imager and biochip sheets are held in an enclosure 210 (only part of which is shown in FIG. 8). The enclosure surrounds the biochips and imager and excludes as much external light from entering the enclosure as possible. A door (not shown) that can be opened and closed provides access to the support 120.

The imager 110 creates data in the form of images to be analysed to detect artefacts. This is carried out using a processor 220 (or computer) held, in the example shown in FIG. 8, in an electricals compartment 230 underneath the support 180.

While the imager 112 could be moved manually on the XY gantry 200 to move the field of view to each biochip face 122 to be imaged (i.e. by the user specifying the gantry position for each move), in various examples, the movement of the imager on the XY gantry is automated. The automation in some examples extends to detecting the position of the field of view relative to a biochip sheet 160 with biochip face to be imaged and to calculating the movement path to be followed by the imager in order to image the biochip faces to be imaged. However, in the examples disclosed in the figures, only the calculation of the movement path to be followed by the imager and subsequent movement is automated. The step of identifying the position of the field of view relative to the biochip sheet and allowing the movement path to be calculated, also referred to as calibrating, is carried out by a user.

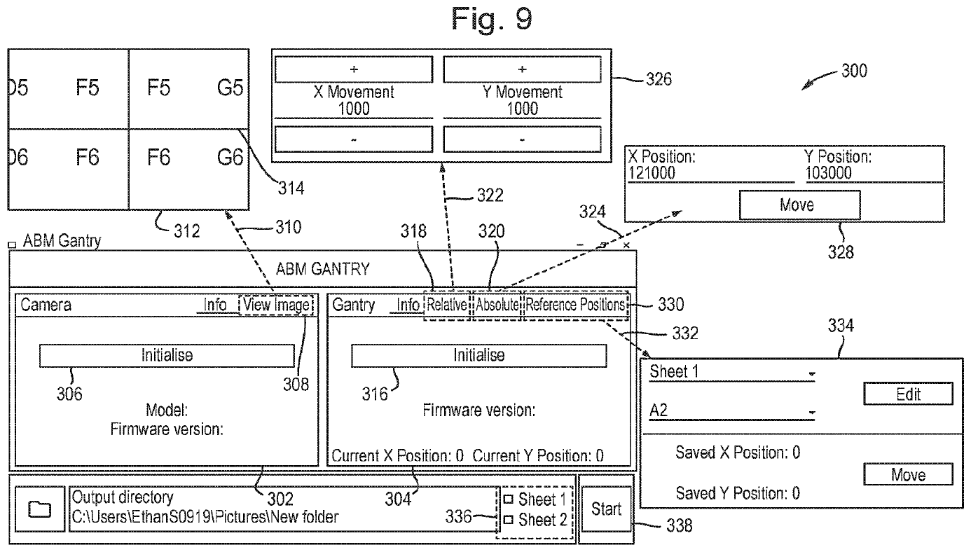

A user calibrates the imager and allows the vision system (or a processor within the vision system) to calculate a movement path using a graphical user interface (GUI) as generally illustrated at 300 in FIG. 9. The GUI allows control of the camera 112, control of the XY gantry 200 and control over whether biochip faces 122 on one biochip sheet 160 or two biochip sheets are to be imaged. As such, the GUI acts as a controller, or at least a user interface of a controller, for the vision system 100.

In FIG. 9 the GUI 300 is shown as being split into a camera section 302 and an XY gantry section 304. Controls for each of the camera and XY gantry are located within the relevant section.

The user is able to control the camera 112 to allow an image to be viewed of the current field of view of the camera. This is achieved by a user clicking the “Initialise” button 306 in the camera section. This causes the camera to start imaging. The user then clicks a “View Image” button 308 in the same section of the GUI 300. This causes the GUI to provide, as indicated by arrow 310, an image 312 of the current field of view 118 of the camera. In addition to the current field of view, a reticule 314 is overlaid on the image. In the example shown in FIG. 9, the reticule is a cross with the intersection of the arms of cross aligning with the centre of the field of view.

Control of the imager position is able to be carried out through two means in the example GUI 300 shown in FIG. 9. First however, the user clicks the “Initialise” button 316 in the XY gantry section 304 to start operation of the gantry. The user then takes further action.

The XY gantry section 304 of the GUI 300 has a “Relative” 318 button and an “Absolute” button 320. As indicated by arrows 322 and 324 in FIG. 9, should the user click on one of these buttons, a window opens.

The window 326 that opens when the “Relative” button 318 is clicked allows a user to move the imager 110 on the XY gantry 200 by a positive or negative distance in the X and/or Y direction relative to the current position of the gantry. In the example shown in FIG. 9 this is achieved using increase and decrease buttons for X and Y. In some examples, an X and/or Y distance for the imager to be moved may also be typed in to the relevant axis direction. Once the movement distance is set, the XY gantry moves the imager by the corresponding amount.

The window 328 that opens when the “Absolute” button 320 is clicked allows a user to move the imager 110 on the XY gantry 200 to specific X and/or Y positions relative to a pre-programmed home position. The home position corresponds to an X position of 0 and a Y position of 0 of the XY gantry. Once the X and/or Y position are input by a user, which is achieved through typing in the example shown in FIG. 9, the user clicks a “Move” button in the window, and the XY gantry moves the imager to the corresponding position.

When the XY gantry 200 moves the imager 110 to a new position, regardless of whether the imager was moved to that position using the relative or absolute mechanism, the imager acquires an image once the movement is completed. This image is then able to be displayed to a user to allow the position to be checked.

The XY gantry section 304 in the example shown in FIG. 9 has a further button. This is a “Reference Positions” button 330. On clicking this button, as illustrated by arrow 332, a window 334 for setting a reference position is opened. This allows a user to select a biochip sheet 160 and the coordinate for one biochip 120, such as “Sheet 1” and “A2” respectively (as shown in the example shown in FIG. 9).

If an input reference point has been set previously, then the X and Y position of for that reference point are displayed in the window. If the input reference point has not been set previously, or needs to be re-set, the reference point can be set to the current position of the gantry, or the gantry can be moved to the appropriate position and the reference point set using the buttons in the window.

In order to align the imager 110 with a desired biochip 120 the position of the XY gantry 200 is moved using the relative or absolute movement mechanism to align the reticule 314 overlaid on the current field of view image 312 with the bottom right corner of the relevant biochip. As such, if, as shown in FIG. 9, biochip E5 is to be set as a reference point, the reticule will be aligned with the join between biochips at E5, F5, E6 and F6. The user is then able to save this reference point by inputting the appropriate information in the window 334 that opens when the user clicks the “Reference Positions” button 330.

By applying this process, a start position and end position for movement of the imager 110 are able to be set. Typically, all the biochips 120 on a biochip sheet 160 are imaged. This means the start position is set as the position for the biochip at A2 and the end position is set for the biochip at 110 for each biochip sheet to be imaged. In other examples other start and end point for respective biochips may be different from each other and/or may be a sub-set of the biochips on the biochip sheet.

Once a start position and an end position and the respective biochip coordinates for those positions are set, the vision system 100 calculates the movement path of the imager 110 in order to allow all the biochips between the start position and end position to be imaged. The calculation takes account of the size of the field of view on the biochip sheet 160, which in some examples is pre-programmed, and in other examples is provided as an input by the user as part of the setup process, or is automatically detected, in addition to the start position and end position and the coordinates of the start position and end position, which provide an indication of how many biochips there are to image due to the biochips being arranged in a grid. This process is carried out for each biochip sheet to be imaged.

As mentioned above, it is possible to image a single biochip sheet 160, or two biochip sheets during a single run of the imager 110. In order to set which biochip sheet is to be imaged, the GUI 300 has selection boxes 336 to allow a user to select which biochip sheets are to be imaged. By selecting that a single biochip sheet is to be imaged, the imaging zone 140 is restricted to the relevant biochip sheet. When two biochip sheets are selected for imaging, the imaging zone is extended across both biochip sheets, or the imaging zone is split into two sections.

Once the start and end positions and reference positions for each biochip sheet to be imaged are set, the user is able to start the imaging process. This is achieved by the user clicking the “Start” button 338 on the GUI.

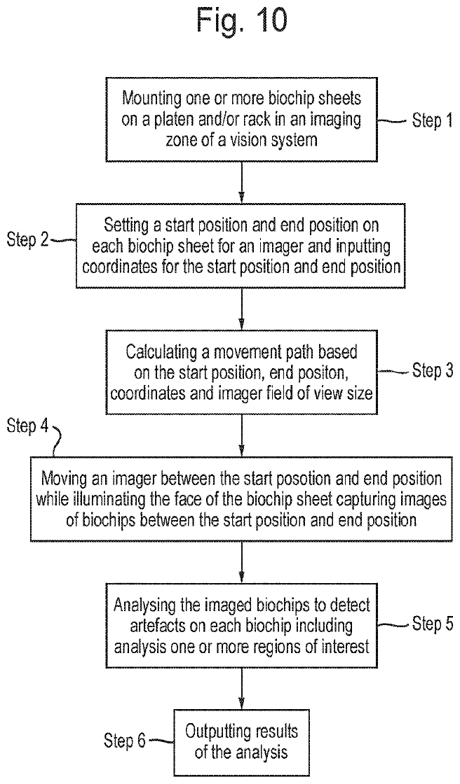

An example process applied to allow biochips to be imaged is shown in FIG. 10. At step 1, biochip sheets to be imaged are mounted into the vision system. This can be on a platen and/or in a rack. As mentioned above, the platen and/or rack is mounted on to a support beneath an imager attached to an XY gantry. This places the biochip sheet(s) in an imaging zone where an imager is able to image the faces of each biochip on the biochip sheet(s).

When loading a biochip sheet on a platen into the vision system, this is typically transferred straight from an automated biochip manufacture (ABM) production line. When loading a biochip sheet on a rack into the vision system, the biochip sheet is placed into the rack prior to mounting the rack in the vision system. Typically a rack is only loaded into the vision system when it is full.

Once the biochip sheet(s) are mounted into the vision system, at step 2, a start position and end position for the imager for each biochip sheet to be imaged is set by applying the processes set out above. Coordinates on the respective biochip sheet for the start and end positions are also set using these processes.

Following the setting of the start and end positions and coordinates, at step 3, the movement path of the imager for each biochip sheet is calculated based on the set start position, end position, coordinates and the imager field of view size.

Imaging of the biochips on each biochip sheet to be imaged is then started at step 4. This includes moving the imager between the start position and end position capturing images of all the biochips between the start position and end position. This is achieved by moving the imager to a suitable position to capture an image of one or more biochips, holding the imager stationary (i.e. in a fixed position on the gantry) when capturing each image, and moving the imager to a new position to image one or more further biochips. During this step the face of the biochip sheet is illuminated. Should the vision system be set to image more than one biochip sheet, the imager is moved between biochip sheets by the XY gantry at a suitable time while imaging is taking place.

At step 5, the images obtained are analysed to detect artefacts on each biochip. The analysis typically includes assessing change in grey value and/or contract between adjacent pixels. In some examples the analysis is carried out by analysing separate areas of each biochip. This is able to be achieved by the system applying one or more regions of interest to the image of each imaged biochip. One region of interest is typically the area of the mask material layer and a second region of interest is typically the area of the discrete reaction zones. These may be applied by the system applying a mask to obscure or ignore the parts of the imaged biochip outside of the region of interest. This may be achieved using typical image processing techniques.

The duration of steps 4 and 5 combined is about 10 seconds to about 20 seconds, with the whole process taking about one minute per biochip sheet.

The one minute duration for the whole process approximately matches the speed at which a biochip sheet is fabricated using the ABM process. As such, once a biochip sheet is fabricated it can be moved immediately to the vision system to be analysed and then removed and replaced with the next biochip sheet. This continuous flow allows a high throughput of biochip sheets.

As a comparison, known systems using a vision system, such as that shown in FIG. 1, instead apply a batch process where a plurality of biochip sheets are loaded in a rack and are each analysed sequentially. The imaging process still takes about 10 seconds to about 20 seconds. However, the process of loading the biochip sheets into the rack takes about one minute per biochip sheet. As such, the example process of FIG. 10 using a vision system such as the example visions system of FIGS. 3 and 8 provides a faster throughput of biochip sheets, thereby increasing the rate at which biochip sheets can be produced and quality controlled.

The results of the analysis are then output at step 6. The results can be reviewed by a user or assessed automatically to identify whether any changes to the ABM process is needed to improve quality of biochips being produced. Additionally, the results output allows one or more biochips or a biochip sheet to be passed or failed on a quality control measure based on the number, variety and/or type of artefacts detected. In some examples, the results include a quality score and/or quality control pass or fail indication, which is produced by the systems comparing the biochips of a single biochip sheet to a pre-determined standard or quality control pass/fail threshold.

1. A vision system for assessing defects on biochips, the vision system comprising:
an imaging region in which a biochip sheet including at least one biochip is locatable in use;
an imager arranged in use to image at least a portion of the imaging region, wherein, when the biochip sheet is located in the imaging region, the portion includes at least a face of the biochip sheet; and
an illumination source arranged in use to direct illumination on to the face of the biochip sheet, thereby illuminating defects on the at least one biochip when a mask material layer is present on the at least one biochip and when a mask material is absent and allowing the defects to be included in the imaged at least a portion of the imaging region.

2. The vision system according to claim 1 further comprising an analyser arranged in use to detect artefacts located on an imaged biochip or at least a portion of the biochip sheet based on an image output from the imager.

3. The vision system according to claim 2, wherein the analyser is arranged in use to detect artefacts by assessing changes in contrast and/or changes in pixel intensity between adjacent pixels in an image.

4. The vision system according to claim 2, wherein the analyser is further arranged in use to apply a region of interest to each imaged biochip or each at least a portion of the biochip sheet and to detect artefacts within the region of interest.

5. The vision system according to claim 4, wherein each biochip of the biochip sheet includes a substrate comprising a mask material layer and a plurality of discrete reaction zones, each zone being an area of the substrate where the mask material is absent, the region of interest applied by the analyser overlapping with at least a part of the mask material layer.

6. The vision system according to claim 2, wherein the analyser is further arranged to output results of the artefact detection for each imaged biochip or each at least a portion of the biochip sheet.

7. (canceled)

8. (canceled)

9. The vision system according to claim 1, wherein the imager is arranged in use to provide a spatial resolution of between about 10 microns (μm) and about 1 μm and the spatial resolution is arranged in use to be provided by one or more of the relative position of the imager and biochip sheet or imaging region, the imager field of view, and size of a sensor of the imager.

10. (canceled)

11. The vision system according to claim 1, wherein the imager is arranged in use to image the portion by imaging individual sections of the portion sequentially such that when the biochip sheet includes a plurality of biochips, the imager images faces of a subset of the plurality of biochips when imaging each individual section of the portion.

12. The vision system according to claim 1, wherein the imager is moveable in use.

13. (canceled)

14. (canceled)

15. The vision system according to claim 12, wherein the position of the imager is adjustable by a user and the imager being arranged in use to provide an image to the user, the image showing the content of the field of view of the imager and a reticule in a fixed position relative to the imager thereby allowing the user to determine the position of the imager relative to the content in the field of view of the imager.

16. The vision system according to claim 12, wherein when imaging the biochips, the imager is arranged in use to travel along a movement path, travel along the movement path causing the imager field of view to be moved to each biochip to be imaged.

17. The vision system according to claim 16, wherein the vision system is arranged in use to calculate the movement path based on a start position and an end position and a value indicative of a number of biochips to be imaged based on the biochips being arranged in an array pattern

18. (canceled)

19. The vision system according to claim 1, wherein the illumination source is a ring light located around an aperture though which the imager is able to obtain images.

20. The vision system according to claim 1 further comprising a support arranged in use to support a platen for a biochip sheet, the platen being locatable to position the biochip sheet in the imaging region.

21. The vision system according to claim 20, wherein the support is further arranged in use to support a rack for biochip sheets, the rack also being locatable to position at least a biochip sheet held at a top of the rack in the imaging region.

22. A method of imaging biochips suitable for assessing defects on biochips, the method comprising:
illuminating, in an imaging region, a biochip sheet including at least one biochip, the illumination being directed on to a face of the biochip sheet; and
imaging at least a portion of the imaging region, the portion including at least a part of the face biochip sheet so as to image a face of at least one biochip of the biochip sheet, thereby illuminating defects on the at least one biochip when a mask material layer is present on the at least one biochip and when a mask material is absent and allowing the defects to be included in the imaged at least a portion of the imaging region.

23. The method according to claim 22 further comprising detecting artefacts located on an imaged biochip by analysing an image of the at least a portion of the imaging region.

24. The method according to claim 23 further comprising outputting results of the artefact detection.

25. The method according to claim 22, wherein the imaging at least a portion of the imaging region includes imaging the face of each biochip to be imaged by moving an imager along a movement path, the movement path being configured to move a field of view of the imager over the face of each biochip to be imaged.

26. The method according to claim 25 further comprising calculating the movement path based on a start position, end position and a value indicative of a number of biochips to be analysed based on the biochips to be imaged being arranged in an array pattern.
